SEQUENCE LISTING (3 sequences)

SEQ ID NO: 1 - DNA, 21 bp, Artificial sequence
  Synthetic
atccccagccggttctgacat

SEQ ID NO: 2 - DNA, 20 bp, Artificial sequence
  Synthetic
tgtcatgctggtctgccgcc

SEQ ID NO: 3 - RNA, 21 nt, Artificial sequence
  Synthetic
aacuucgacuuugucaccgag

(19) United States
(12) Patent Application Publication
(10) Pub. No.: US 2013/0004494 A1
(43) Pub. Date: Jan. 3, 2013

(54) Inhibition of P21 and Use Thereof for Inducing Tissue Regeneration

(76) Inventor: Ellen Heber-Katz, Philadelphia, PA (US)

(21) Appl. No.: 13/583,777

(22) PCT Filed: Mar. 11, 2011
(86) PCT No.: PCT/US11/28130

§ 371 Date: Sep. 10, 2012

Related U.S. Application Data
(60) Provisional application No. 61/313,448, filed on Mar. 12, 2010.

(51) Int. Cl. A61K 39/395; A61P 43/00; C07K 16/18

(52) U.S. Cl. 424/135.1; 530/387.3

(57) ABSTRACT

The present invention embraces a method for inducing tissue regeneration using a p21 inhibitor as well as a bio-compatible tissue engineering product containing a p21 inhibitor.

### INTRODUCTION

This application claims the benefit of priority of U.S. Provisional Application No. 61/313,448, filed Mar. 12, 2010, the content of which is incorporated herein by reference in its entirety.

This invention was made with government support under Grant No. R01GM073226 awarded by the National Institutes of Health. The government has certain rights in the invention.

### BACKGROUND OF THE INVENTION

The ability to regenerate appendages is generally considered to be a property of organisms other than mammals, which typically heal wounds by the process of repair characterized by wound site contraction and closure with a scar. On the other hand, the replacement of limbs in the adult newt and the axolotl, for example, after injury or amputation begins with the formation of a blastema, a structure with highly proliferative cells that grows until that appendage is replaced without scarring (Stocum (2004) Curr. Top. Microbiol. Immunol. 280:1-70; Brockes & Kumar (2005) Science 310:1919-23). The ability of blastemal cells in the adult to proliferate, until normal architecture with appropriate differentiation into multiple cell types is achieved, is a defining feature of regeneration.

The MRL mouse and its close relatives (“healer” strains) have unique healing and regenerative capabilities, including the complete closure and tissue regeneration of through-and-through ear-hole puncture wounds with the formation of a circular blastema (Desquenne-Clark, et al. (1998) Clin. Imm. and Immunopath. 88:35-45), the re-growth of articular cartilage (Fitzgerald, et al. (2008) Osteoarthritis and Cartilage 16:1319-1326), and the partial regeneration of amputated digits (Chadwick, et al. (2007) Wound Repair Regen. 15:275-284; Gourevitch, et al. (2009) Wound Repair Regen. 17:447-455).

Genetic mapping of the healing trait using microsatellite and SNP mapping indicates that the MRL healing phenotype is distributed among over 20 loci on multiple chromosomes (McBrearty, et al. (1998) Proc. Natl. Acad. Sci. USA, 95:11792-11797; Blankenhorn, et al. (2003) Mammalian Genome 14:250-260; Masinde, et al. (2001) Genome Res. 11:2027-2033; Yu, et al. (2005) Mammalian Genome 16:918-924; Blankenhorn, et al. (2009) Mammalian Genome 20:720-733). Mechanisms and insights similar with classical regenerating species such sponges, hydra, planaria, and newt and in cases of limited regeneration (i.e., liver) in mammals are under investigation.

Sponges and hydra are classic model species used in the study of wound healing, regeneration, allograft rejection and innate immunity (Wiens, et al. (2004) Immunogenetics 56:597-610). These organisms belong to two of the oldest metazoan lineages in the fossil record and provide molecular and cellular insight into the first evolutionary strategies used by multicellular animals to repair tissue injury and respond to microbial infection. In the case of sponges, the ability to heal injuries and regenerate lost tissue, and the ability to recognize and reject foreign tissue are both associated with the activation of a DNA damage response characterized by increased single-strand scission and the appearance of ribosubstitution and other alkali-labile sites in DNA (Muller, et al. (2006) Mutat. Res. 597:62-72).

In hydra, cells undergo programmed developmental replacement leading to an apparent indefinite life-span (Martinez (1998) Exp. Geront. 33:217-225). Hydra have a large number of regenerative cells such as interstitial gland cells (Schmidt & David (1986) J. Cell Sci. 85:197-215), epithelial cells (Dubel & Schaller (1990) J. Cell Biol. 110:939-945; Holstein, et al. (1991) Dev. Biol. 148:602-11) and cells in the foot (Ulrich & Tárnok (2005) Cell Prolif. 38:63-75), which have been shown to exhibit a unique cell-cycle phenotype characterized by G2/M bias. Adult urodele amphibians (e.g., the newt), which can regenerate many of their tissues initiate cell cycle re-entry, local dedifferentiation and proliferation (Brockes & Kumar (2005) supra). Moreover, in culture, myotubes derived from the newt show serum stimulation driving the myotubes into S phase and arrest in G2 (Tanaka, et al. (1997) J. Cell Biol. 136:155-165). In the regenerative mammalian liver, G2 arrest is a dominant feature in adult hepatocytes where up to 70% are tetraploid (Michalopoulos & DeFrances (1997) Science 276:60-66). Finally stem cells also show a preference for G2/M arrest (Chuykin, et al. (2008) Cell Cycle 7:2922-2928; Hong, et al. (2007) Mutation Research 614:48-55; Galvin, et al. (2008) Stem Cells 26:1027-36).

### SUMMARY OF THE INVENTION

The present invention features a method for inducing tissue regeneration by administering to the tissue of a subject in need of treatment an effective amount of a p21 inhibitor. In one embodiment, the p21 inhibitor directly inhibits p21 activity. In another embodiment, the p21 inhibitor inhibits expression of p21.

A biocompatible tissue engineering product containing a p21 inhibitor is also provided.

### DETAILED DESCRIPTION OF THE INVENTION

Many mammals including humans and most mouse strains are capable of tissue regeneration to varying degrees. This ranges from the replacement of extensively ressected liver lobes to the interstitial replacement of damaged skeletal muscle cells, epithelium, the gut lining, and a modest life-long replacement of CNS neurons and cardiomyocytes. In contrast, with few exceptions (ear hole closure in rabbits and seasonal antler replacement), the regeneration of lost appendage tissue is virtually never seen. It has now been shown that the MRL mouse strain (and close relatives), unique among mice in their ability to close ear holes, show high levels of DNA damage, a G2/M bias, and a lack of p21 protein expression in both uninjured steady state tissue and post-injury. The functional role of p21 was demonstrated in a p21 knockout mouse, which displays the same range of cellular effects as seen in the MRL mouse and reproduces appendage regeneration in vivo in whole animals. Accordingly, the present invention provides methods for inducing tissue regeneration using a p21 inhibitor. Such tissue generation is of use in repairing wounds or defects in skin or other tissues and in inhibiting excessive scar formation.

According to the present invention, a sufficient dose of a p21 inhibitor is administered to the tissue of a subject (e.g., a patient) in need of such treatment. A subject “in need of such treatment” can be, e.g., a subject with a wound; damaged/injured organ or tissue (e.g., skin or muscle); and/or tissue or organ defect, wherein administration of a p21 inhibitor induces or facilitates repair and/or regeneration of said tissue or organ. Tissue regeneration in the context of the present invention includes in vivo, in vitro or ex vivo applications of tissues, with particular embodiments embracing regeneration of tissues which do not normally regenerate. Desirably, tissue regeneration is induced locally at the site of administration. In this respect, a p21 inhibitor can be administered locally to a wound site of a subject to induce tissue regeneration by biological interaction with surrounding tissues. As used herein, “induce”, as well as the correlated terms “induction” and “inducing”, refer to the action of generating, promoting, forming, regulating, activating, enhancing or accelerating a biological phenomenon. Subjects benefiting from treatment in accordance with the method of this invention include mammals such as rats, mice, rabbits, dogs, cats, goats, sheep, cows, pigs, primates and humans.

Tissues that can be treated using methods of the invention include, but are not limited to, those with cuts, stretches, tears, pulls, abrasions, burns, bone breaks, crushes, scrapes, contusions, bruises, and the like. Particularly, peripheral or central nerve injuries, such as crushed or severed nerves, including the spinal cord, can be treated. Methods and compositions of the invention can be used to treat and thus enhance healing of a tissue by promoting processes such as angiogenesis, chondrogenesis, return of hair follicles and/or sebaceous glands, reepithelialization, rapid connective tissue proliferation, deposition of organized extracellular matrix, and restoration of normal tissue architecture and function. Surgical adhesions can be prevented by prophylactic treatment of surgical incisions using compositions and methods of the invention. These methods and compositions are useful in any situation in which regeneration or healing of a wound without formation of scar tissue is desired.

As is conventional in the art, p21 (GeneID: 1026), also known as cyclin-dependent kinase inhibitor 1A (CDKN1A), binds to and inhibits the activity of cyclin-CDK2 or —CDK4 complexes, and functions as a regulator of cell cycle progression at G1. The expression of this gene is tightly controlled by the tumor suppressor protein p53, through which this protein mediates the p53-dependent cell cycle G1 phase arrest in response to a variety of stress stimuli. This protein can interact with proliferating cell nuclear antigen (PCNA), a DNA polymerase accessory factor, and plays a regulatory role in S phase DNA replication and DNA damage repair. This protein was reported to be specifically cleaved by CASP3-like caspases, which thus leads to a dramatic activation of CDK2, and may be instrumental in the execution of apoptosis following caspase activation.

Based upon the observations herein, without the p21 protein, a G1 checkpoint cannot be fully enacted, leading to a dependency on the G2 checkpoint, which explains the remarkable cell cycle profiles of healer cells. Furthermore, without p21, un-scheduled entry into S-phase occurs and enhanced proliferation is seen. In this respect, studies examining p21 deletion in human fibroblasts has shown an up-regulation of p53 with enhanced proliferation and replication stress (Perucca, et al. (2009) Cell Cycle 8:105-114), similar to the regeneration phenotype observed herein. Moreover, increased DNA damage is reported in cells lacking p21, a result likely due to proliferative and replicative stress (Perucca, et al. (2009) supra). The inability to enter quiescence at G0 in response to stress leads to G2 arrest, which is also observed in MRL mice.

The importance of cell cycle control for the regeneration phenotype has been suggested. In vitro regenerative potential is associated with hyper-phosphorylation of Rb in newt myotube cultures (Tanaka, et al. (1997) supra) and in Rb−/− mouse myotubes (Schneider, et al. (1994) Science 264:1467-1471). These two cases would be functionally similar to the absence of p21, where cyclin-cdk interactions would not be inhibited and would thus bypass the G1 checkpoint. In vivo, the absence of p21 has been shown in double mutant mice to both enhance liver regeneration and muscle cell proliferation (Stepniak, et al. (2006) Genes Dev. 20:2306-2314; Willenbring, et al. (2008) Cancer Cell 14:59-67; Hawke, et al. (2003) J. Biol. Chem. 278:4015-20). However, in both cases, these tissues normally regenerate.

Based upon the findings disclosed herein, inhibitors of p21 find application in blocking, attenuating or inhibiting p21 activity, thereby facilitating, enhancing or inducing tissue regeneration. p21 activities that can be inhibited by an agent disclosed herein include, e.g., any biochemical, cellular, or physiological property that results from p21 activity. An effective amount of a p21 inhibitor is an amount that measurably decreases or inhibits a property or biochemical activity possessed by the protein, e.g., the ability to inhibit the activity of cyclin-CDK2 or -CDK4 complexes, or the interaction with PCNA, CUL4A, TSG101, CIZ1, Cyclin-dependent kinase 2, GADD45G, GADD45A, DTL, Thymidine kinase 1, Cyclin E1, PIM1, BCCIP and/or DDB1. In one embodiment, the activity of p21 is directly inhibited. In accordance with this embodiment, the inhibitory agent of the invention specifically interacts with the DNA or RNA encoding p21 and inhibits the transcription or translation of p21, or alternatively interacts with p21 and inhibits the activity of p21. In another embodiment, the inhibitory agent of the invention indirectly inhibits p21 by, e.g., inhibiting p53-mediated expression of p21. In particular embodiments, the p21 inhibitor of this invention is selective for p21 and does not inhibit the activity of other cyclin-dependent kinase inhibitors including, e.g., CDKN1B (GeneID: 1027), CDKN1C (GeneID: 1028), CDKN2A (GeneID: 1029), CDKN2B (GeneID: 1030), CDKN2C (GeneID: 1031), CDKN2D (GeneID: 1032), CDKN3 (GeneID: 1033).

Inhibitors that decrease the expression or activity of p21 desirably provide a 50%, 60%, 70%, 80% or 90% decrease in the expression or activity of p21. Most preferably, effective expression or activity of p21 is decreased by 90%, 95%, 99%, or 100%. Expression or activity of p21 can be assessed using methods well known in the art, such as hybridization of nucleotide probes to mRNA, quantitative RT-PCR, or detection of p21 protein using specific antibodies.

Agents that inhibit the transcription or translation of p21 include, e.g., ribozymes, inhibitory RNA molecules (e.g., siRNA or shRNA), antisense molecules and the like. Such molecules can be derived from the nucleotide sequence encoding p21 (e.g., as disclosed in GENBANK Accession No. NM_{—}000389 (human) or NM_{—}007669 (mouse)) using conventional approaches. Agents that inhibit transcription or translation are typically complementary to at least a portion of the coding sequence or 5′ or 3′ UTR of the gene. Inhibitor agents are generally at least 11 nucleotides in length, but can be at least 12, 15, 20, 25, 30, 35, 40, 45, or 50 or more nucleotides long. Longer sequences can also be used. Exemplary inhibitory RNA molecules of use in the present invention include, but are not limited to, the human p21 antisense oligodeoxynucleotide 5′-ATC CCC AGC CGG TTC TGA CAT-3′ (SEQ ID NO:1; Fan (2003) Mol. Cancer. Ther. 2:773-82); the p21 antisense oligodeoxynucleotide 5′-TGT CAT GCT GGT CTG CCG CC-3′ (SEQ ID NO:2; Liu, et al. (2006) Cell Biol. Internatl. 30:283-287); a siRNA molecule targeting the human p21 sequence 5′-AAC UUC GAC UUU GUC ACC GAG-3 (SEQ ID NO:3), which corresponds to the coding region 148-168 relative to the start codon (Hastak, et al. (2005) FASEB J. 19:789-91); the p21 siRNA (m2) or p21 shRNA plasmid (m2) available from Santa Cruz Biotechnology, Inc. (Santa Cruz, Calif.); or the human-specific SIGNALSILENCE p21 Waf1/Cip1 siRNA available from Cell Signaling Technology (Danvers, Mass.). See also US 2003/0144236 and US 2005/0043262 for exemplary antisense molecules. Inhibitor molecules can be provided in a construct and introduced into cells using standard methodologies to decrease expression of p21.

Exemplary agents that inhibit p21 activity include, but are not limited to, inhibitory proteins or peptides, small organic molecules and antagonistic antibodies. For example, Park, et al. ((2008) Cancer Biol. Ther. 7:2015-2022) describe 12 small molecule inhibitors of p21 identified from a 3-(1,2-disubstituted-1H-benzoimidazol-5-yl)-3-(arylureido/acylamino)-propionamide one-bead-one-compound library, which would be of use in the methods of this invention.

Antibodies which specifically bind to p21 protein can also be used to alter the activity of p21. p21-specific antibodies bind to p21 and prevent the protein from functioning in the cell. Preparations of polyclonal and monoclonal antibodies can be made using standard methods. Antibody fragments such as Fab, single-chain Fv, or F(ab′)₂ fragments can also be prepared. If desired, antibodies and antibody fragments can also be “humanized” to prevent a patient from mounting an immune response against the antibody when it is used therapeutically, as is known in the art. Other types of antibodies, such as chimeric antibodies, can be constructed as disclosed, for example, in WO 93/03151. Binding proteins which are derived from immunoglobulins and which are multivalent and multispecific, such as the “diabodies” described in WO 94/13804, can be prepared and used in methods of the invention. Anti-idiotype antibodies, directed against unique sequence variants, can also be used in therapeutic methods of the invention.

Alternatively, p21 inhibitors can be identified in in vitro or in vivo screening assays that monitor the effect of a compound on the expression or activity of p21. Test agents that can be screened encompass numerous chemical classes, although typically they are organic compounds. In some embodiments, the candidate agents are small organic compounds, i.e., those having a molecular weight of more than 50 yet less than about 2500, preferably less than about 1000 and, more preferably, less than about 500. Candidate test agents generally include functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate test agents can have a cyclic carbon or heterocyclic structure and/or aromatic or polyaromatic structures substituted with one or more of the above-identified functional groups. Candidate test agents also can be biomolecules such as peptides, proteins, antibodies, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

For use in the methods described herein, the p21 inhibitor can be prepared as a pharmaceutical composition suitable for administration to a tissue in need of regeneration. As used herein, a “pharmaceutical composition” is a p21 inhibitor in admixture with a pharmaceutically acceptable carrier. The pharmaceutically acceptable carrier can be, e.g., a solvent, excipient, or matrix used to administer the p21 inhibitor. Pharmaceutical compositions can comprise any solvent, dispersion media, aqueous, gaseous solutions, antibacterial or antifungal agents, isotonic agents, either absorption delayer or accelerator agents, or similar substances. The use of said substances in the administration of pharmaceutically active compositions is known in the art. Supplementary active ingredients may also be incorporated to the pharmaceutical composition utilized in the present invention. Pharmaceutical compositions can include, e.g., inert solid fillings or solvents, sterile aqueous solutions and non-toxic organic solvents. The pharmaceutically acceptable carrier should not react with or reduce in any other manner the efficiency or stability of the p21 inhibitor. Pharmaceutically acceptable carriers include, but are not limited to, water, ethanol, polyethyleneglycol, mineral oil, petrolatum, lanolin, and slowly metabolized macromolecules, such as proteins, polysaccharides, polylactic acids, polyglycolic acids, polymeric amino acids, amino acid copolymers, inactive virus particles and similar agents. In some embodiments, a p21 inhibitor of the invention is formulated such that it is administered under slow-release conditions. Any repeated administration formulation or protocol can be used.

A pharmaceutical composition of the invention also includes a cell that secretes a p21 inhibitor (e.g., a protein- or peptide-based p21 inhibitor). The cells employed can naturally secrete the p21 inhibitor, or they may be genetically engineered to secrete the p21 inhibitor. For example, cells, such as dermal fibroblasts or peripheral blood leukocytes, can be removed from a subject, transfected with the gene encoding a p21 inhibitor, and then be replaced into the same or another mammal with a wound, preferably at or within the vicinity of the wound to enhance healing of the wound. Preferred cells include macrophages, stem cells, fetal liver cells, peripheral blood leukocytes, and bone marrow cells. Extracts from these cells can be prepared using standard methodologies and also used for wound treatment. The cells or cellular extracts can be placed directly at the site of the wound to promote its healing.

A pharmaceutical composition of the present invention can be administered by a variety of routes including local or systemic routes, with particular embodiments embracing local administration to a tissue in need of regeneration. In this respect, the p21 inhibitor can be administered by injection, oral administration, particle gun, catheterized administration, or topical administration. In some embodiments, the pharmaceutical composition of the present invention is moldable or cast into a shaped form. For treatment of wounds on the surface of the body, a wound healing composition is typically prepared in a topical form, either as a liquid solution, suspension, gel, putty, paste or cream. However, solid forms suitable for solution or suspension in liquid vehicles prior to injection can also be prepared, for local treatment of internal wounds. Moreover, administration can be via a synthetic polymer, polymer scaffold, polymer matrix, or wound dressing material. In this respect, the present invention embraces a biocompatible tissue engineering product containing a p21 inhibitor.

A biocompatible tissue engineering product as used herein is a biocompatible material that conforms to the complex shapes of tissue structures requiring repair or reconstruction. Such tissue engineering products are routinely used in the art and are generally composed of a polymerized matrix optionally containing viable cells, enhancers, stabilizers, photoreactors, and the like, that improve the performance, stability and durability of the product for use in vivo, particularly for reforming degenerated, damaged or diseased tissue.

The dose of p21 inhibitor for any particular use will vary from subject to subject, depending on, e.g., the species, age, weight and general or clinical condition of the subject, the particular agent or vehicle used, the method and scheduling of administration, and the like. A therapeutically sufficient dose can be determined empirically, by conventional procedures known to those of skill in the art. See, e.g., The Pharmacological Basis of Therapeutics, Goodman and Gilman, eds., Macmillan Publishing Co., New York. For example, a sufficient dose can be estimated initially either in cell culture assays or in suitable animal models. The animal model may also be used to determine the appropriate concentration ranges and routes of administration. In this respect, a p21 inhibitor can be administered to a model, such as a rat or mouse, which has a wound, and tissue regeneration can be determined. The wound can be, for example, a cut or abrasion in the skin, a tail or ear cut or an ear punch, a cut in the liver, or a severed or crushed nerve, including an optic nerve or spinal cord. Such information can then be used to determine useful doses and routes for administration in humans.

Properties of a wound healing model which can be assessed include, but are not limited to, enhanced wound healing, enhanced tissue regeneration, cell growth, apoptosis, cell replication, cell movement, cell adhesion, DNA synthesis, protein synthesis, mRNA synthesis, and mRNA stability. Methods of assessing these properties include morphological assessment, either with or without the aid of a microscope, as well as biochemical and molecular biology methods well-known in the art.

The invention is described in greater detail by the following non-limiting examples.

### Example 1

### Materials and Methods

Animals.

Commercially available mice were obtained from Jackson or Taconic Laboratories. Through-and-through ear hole punches were carried out according to known methods (Desquenne-Clark, et al. (1998) supra).

Cell Culture, Cell Cycle Analysis and Comet Assays.

Primary skin dermal cells from ear pinnae were established and cells from early passages were used. These cells were analyzed for cell cycle using either Propidium Iodide (PI) dye (Sigma, St. Louis, Mo.) or VYBRANT DYECYCLE Orange (Molecular Probes, Eugene, Oreg.) by flow cytometry. Comets were prepared using these cells according to known methods (Speit & Hartmann (2006) Methods Mol. Biol. 314:275-286).

Immunohistochemistry.

Primary ear-skin dermal cells were grown on glass coverslips and tissue from normal ears and from the small intestine were fixed and embedded. Staining with antibody was according to conventional methods (Gourevitch, et al. (2009) supra).

Western Blot Analysis.

Western blot analysis was carried out for four different antigens (p53, γH2AX, caspase 3, p21, and actin as a control) using three different tissues (cultured dermal cells, ear pinnae, and small intestine).

TUNEL Analysis.

Paraffin-embedded small intestine was analyzed using the DERMATACS In Situ Apoptosis Detection Kit (Trevigen, Inc., Gaithersburg, Md.).

### Example 2

### G2/M Accumulation in Cells from Healer Mice

Since models of tissue regeneration have implicated cell cycle control mechanisms as a potential common feature among cells capable of tissue regeneration, the cell cycle profile of cells cultured from normal uninjured ear tissue derived from adult healer mice were examined. For this purpose, healer MRL and LG/J mice, a congenic line selected for healing, healer and non-healer recombinant inbred (RI) lines generated from LG/J healer and SM/J non-healer mice (Hrbek, et al. (2006) Mammalian Genome 17:417-429), and non-healer BE and SM/J mice were used. MRL shares 75% of its genome with LG/J, having been produced by two final backcrosses to LG/J (Murphy & Roths (1979) In Genetic Control of Autoimmune Disease. Ed. NR Rose, Bigazzi, and Warner (Elsevier, New York) p. 207-220). The cell cycle profile from the in vitro cultured cells of MRL healer and related strains were analyzed to determine whether the profiles were different from control non-healer mice. Using standard propidium iodide DNA content labeling and flow cytometry analysis, cell cycle profiles were compared, and the healer cells showed a definitive accumulation in the G2/M phase versus control cells. Four different pairs of cells were used and all showed a similar accumulation. The most dramatic differences were observed when the RI healer and non-healer strains and the congenic and B6 strains were compared. In the non-healers, the profile was consistent with what is generally observed in a normal mouse cell population, in that the majority of cells were in G1 phase, with smaller percentages seen in S-phase and G2/M. In contrast, the healer cells and in particular the RI line 6 showed 21.5% of cells in G0/G1 while 64.1% of cells were in G2/M phase. The pronounced accumulation of cells in G2/M was consistent with that observed in other models of regeneration suggesting that cell cycle control mechanisms are associated with cellular regenerative potential.

### Example 3

### Expression and Stabilization of p53 in MRL Healer Cells

The G2/M transition is regulated by a complex series of molecular interactions that can elicit a cell cycle checkpoint that may involve the tumor suppressor p53 protein. To determine if p53 was involved, two different methods were used to assess p53 expression levels. First, in vitro experiments were carried out with MRL and congenic cells derived from normal uninjured ear tissue compared to non-healer B6 cells for steady state levels of p53 protein. MRL and healer congenic cells had readily detectable levels of p53, while little or no p53 was detected in B6. By FACS analysis, it was also found that most of the p53-positive cells were in the G2/M stage of the cell cycle.

Healer and nonhealer tissues were also examined. Histological sections from normal uninjured MRL and B6 ear tissue and small intestine were processed for immuno-histochemistry (IHC). p53 expression was observed in normal MRL tissue, but was lower or not detectable in B6 tissue. This was supported by western blot analysis of normal ear tissue.

To further explore the role of p53 during the regenerative process in vivo, ear hole closure assays were performed on B6 and MRL mice. Extracts from healing ear tissue on day 0, 5 and 10 post injury were monitored for p53 expression using western blot analysis. p53 protein levels were increased before and during healing in tissue from MRL compared to B6, showing that this increase in expression accompanies the healer phenotype. These data indicate that a cell cycle checkpoint response was active at steady state and during healing.

### Example 4

### Spontaneous DNA Damage Foci in MRL Healer Mice

The DNA damage response cascade is enacted by two large kinases, ATM and ATR, which respond to various cellular stresses. An early hallmark of an active DNA damage response is the phosphorylation of the variant histone H2AX on the serine 139 residue or (γH2AX) (Rogakou, et al. (1998) J. Biol. Chem. 273:5858-5868). Using immunofluorescence, in vitro cultured MRL and congenic cells derived from normal uninjured ear tissue showed greater numbers of γH2AX foci than control B6 cells, including higher numbers of foci-positive cells and higher numbers of foci per cell. As a control, γ-irradiated (1 Gy) cells showed increased γH2AX foci after irradiation.

Tissue from uninjured ear and small intestine was also examined by IHC. MRL tissue displayed a greater level of γH2AX staining compared to B6 tissue. Protein extracts from cultured ear cells and chromatin-enriched ear tissue also demonstrated high levels of γH2AX levels by western blot analysis, reaffirming an active DNA damage response in MRL and congenic normal uninjured cells, both in vitro and in vivo.

The histone protein H2AX can be phosphorylated proximal to DNA double strand breaks (DSB) after exposure to clastogenic agents such as ionizing radiation, but also after replication-associated DSB that occur when gaps or single-stranded regions are present in front of an advancing replication fork. The DNA damage response pathway that generally governs and protects against so-called replication stress is maintained by the ATR kinase. This kinase is activated by the TopBP1 protein (Kumagai et al. (2006) Cell 124:888-890). The normal uninjured ear-derived cells were analyzed for increased TopBP1 foci by IHC to further determine an association with an active replication stress response. Like γH2AX, TopBP1 foci were markedly enriched in the healer cells, indicative of an active and constitutive DNA damage checkpoint.

### Example 5

### Cells from Healer Mice Display Elevated Levels of Endogenous DNA Damage

Numerous cellular stresses converge upon p53 activation and DNA damage response pathways. To test for bona fide DNA damage, a comet assay was performed under both neutral conditions (detecting DSB alone) and alkaline conditions (detecting both single strand breaks (SSB) and DSB). The alkaline comet results showed high numbers of comets from MRL and other healer cells derived from normal uninjured ear tissue (an average of 80% comet positive) with large tail moments. This is in contrast to few or no comets in non-healer cells. These results agree with the γH2AX foci findings. The neutral comet assay (DSB only) counts were lower than the alkaline (DSB and SSB) counts, but still averaged 35% comet-positive cells in the healers.

### Example 6

### Increased Rad51 Foci in Healer Cells

Cells involved in regeneration of tissue should be proliferation-competent and the endogenous DNA damage observed must be repaired. There are two major pathways responsible for DNA DSB repair. Non-homologous end joining can occur throughout the cell cycle but may be error prone. Homologous recombination, though, is error free and uses a sister chromatid as a template for repair during late S or G2 phases of the cell cycle. Since a G2/M accumulation of MRL cells was observed, it was determined whether homologous recombination pathways were up-regulated to repair the endogenous damage. The results of this analysis indicated that Rad51 foci increased in 10-15% of in vitro cultured healer cells compared to 1-2% non-healer cells.

### Example 7

### Healer Cells In Vivo have Increased Markers of Apoptosis

While many of the healer cells are repairing the intrinsic DNA damage, others clearly have a different outcome. To examine apoptosis, caspase 3 and TUNEL were examined. MRL tissue showed increased caspase 3 by western blot analysis using extracts from small intestine and IHC staining of uninjured ear tissue. An increase in TUNEL-positive cells in MRL small intestine was also seen. While the regeneration process requires cell proliferation, it appears to come at a cost to many cells in the population as markers of apoptosis were distinctly increased in healer cells.

### Example 8

### Lack of p21 Expression/Induction in Healer Cells

The pronounced intrinsic DNA damage in healer cells was strikingly consistent with reports showing that mouse embryonic stem cells display endogenous DNA damage and a faulty G1 checkpoint (Galvin, et al. (2008) supra; Hong & Stambrook (2004) Proc. Natl. Acad. Sci. USA 101:14443-14448). In those reports, a lack of checkpoint control was attributed in part to a lack of p21 induction. Accordingly, it was determined whether the MRL healer cells also failed to induce the p21 checkpoint protein. In comparison to both a human cell line (HCT116) and a mouse non-healer cell line (B6), MRL cells did not display p21 protein expression and furthermore, expression was not induced after DNA damage following ionizing radiation.

### Example 9

### Deletion of p21 Converts a Non-Healer into a Regeneration-Competent Healer Mouse

Since p21 was down-regulated in healer cells, it was determined whether the deletion of p21 would permit appendage regeneration potential in vivo in a non-regenerating mouse. CDKN1A (p21) −/− mice (Brugarolas, et al. (1995) Nature 377:552-557) were examined for ear hole closure over a one month period. The background control strain B6129SF2/J mice were ear punched in parallel and the data compared to MRL and B6 ear hole closure. This analysis indicated that ear hole closure in 6-7 week old p21 −/− mice was almost identical to that seen in MRL healer mice, healing only slightly less well than MRLs on day 28. Age-matched p21 +/+ controls did not close and were similar to B6 non-healer mice. Gross and histological analysis of the p21 −/− ear holes over time showed closing holes with normal dermis, epidermis, and cartilage interspersed with thickened areas of dermis. To further establish the link between G1 checkpoint control and the regeneration phenotype, cells derived from the ear pinnae of normal uninjured p21+/+ and p21−/− mice were examined for DNA damage and cell cycle arrest. γH2AX staining, comet formation, and increased levels of cells in G2/M were found in the p21−/− mice compared to the p21+/+ mice. This directly correlated with what was seen in the healer MRL, congenic, and RI mouse cells described above in vivo and in vitro, solidifying the role of p21 under-expression and tissue and appendage regeneration.

What is claimed is:

1. A method for inducing tissue regeneration comprising administering to tissue of a subject in need of treatment an effective amount of a p21 inhibitor thereby inducing tissue regeneration.

2. The method of claim 1, wherein the p21 inhibitor directly inhibits p21 activity.

3. The method of claim 1, wherein the p21 inhibitor inhibits expression of p21.

4. A biocompatible tissue engineering product comprising a p21 inhibitor.
